(19) United States
(12) Patent Application Publication
(10) Pub. No.: US 2021/0147101 A1
(43) Pub. Date: May 20, 2021

(54) APPARATUS AND METHOD FOR MANUFACTURING STICK CONTAINER

(71) Applicant: ENC CO., LTD., Yongin-si (KR)

(72) Inventors: Kwang Hyun SHIN, Suwon-si (KR); Seung Gyu PARK, Paju-si (KR)

(21) Appl. No.: 16/951,235

(22) Filed: Nov. 18, 2020

(30) Foreign Application Priority Data
Nov. 18, 2019 (KR) 10-2019-0147912

(51) Int. Cl. B65B 9/20; B65B 51/26; B65B 51/32; B65B 9/02

(52) U.S. Cl. CPC B65B 9/20; B65B 51/26; B65B 2220/24; B65B 9/023; B65B 51/32

(57) ABSTRACT

An apparatus for manufacturing a stick container having a pair of filler accommodating parts by processing a sheet-shaped material, the apparatus comprises: a lengthwise sealing unit that bonds side surfaces of the material to each other in order for the material to have an internal space which is elongated in a lengthwise direction; a widthwise sealing unit that bonds the material to separate the internal space along the lengthwise direction; and a filling unit that is configured to fill a first filler and a second filler in the separated internal spaces, respectively, wherein the widthwise sealing unit includes: a top and bottom surface sealing unit that bonds a bottom surface of the stick container where the first filler is to be filled and bonds a top surface of the stick container where the second filler has been injected; and a separation surface sealing unit that bonds a central portion of the stick container after the first filer is filled and before the second filler is filled.

### CROSS-REFERENCE TO RELATED APPLICATION

This application claims the benefit under 35 USC 119(a) of Korean Patent Application No. 10-2019-0147912 filed on Nov. 18, 2019 in the Korean Intellectual Property Office, the entire disclosures of which are incorporated herein by reference for all purposes.

### TECHNICAL FIELD

The present disclosure relates to an apparatus and method for manufacturing a stick container configured to accommodate cosmetics or the like.

### BACKGROUND

As the interest in skin care increases, the use of nourishing masks for facial skin care is being increased. In general, a nourishing pack refers to a pack in which active ingredients or cosmetics containing various components effective in moisturizing, whitening and nourishing skin are contained.

In recent years, products that are manufactured by injecting contents different in ingredient and effect into a single pack container and can be used by mixing the contents right before use have come out. If the contents are mixed and then injected into the pack container, the efficacy may be decreased. Therefore, the contents are mixed right before use to maximize the efficacy.

To manufacture such a pack container, if a worker fills contents into separate internal spaces, respectively, of a pack container, it is difficult to fill an adequate amount of contents into the pack container. Also, the filing operation requires a lot of people and time. The pack container can be manufactured into various shapes such as a square or stick shape (bar shape).

Besides, the pack container may be used as a container that contains beverage, coffee, tea or other instant food. An adequate amount of food, for example, a mixture of coffee, sugar and/or creamer for one serving, may be contained in a container. For another example, if an instant food is made of contents which are different in properties and efficacy and individually separated in a container, adequate amounts of the separated contents can be mixed by applying pressure to the container right before eating, so that any one can eat the contents mixed as he/she prefers.

### SUMMARY

An object of the present disclosure is to provide an apparatus and method for manufacturing a stick container by injecting contents different in component and effect into a first filling part and a second filling part, respectively, of a single pack container and packing them in order for a user to mix and use the contents right before use.

Another object of the present disclosure is to provide an automated apparatus and method for supporting to manufacture a stick container, which is filled with different kinds of contents in respective separate internal spaces, in great numbers at once.

However, the problems to be solved by the present disclosure are not limited to the above-described problems. There may be other problems to be solved by the present disclosure.

According to an aspect of the present disclosure, an apparatus for manufacturing a stick container having a pair of filler accommodating parts by processing a sheet-shaped material, the apparatus comprises: a lengthwise sealing unit that bonds side surfaces of the material to each other in order for the material to have an internal space which is elongated in a lengthwise direction; a widthwise sealing unit that bonds the material to separate the internal space along the lengthwise direction; and a filling unit that is configured to fill a first filler and a second filler in the separated internal spaces, respectively, wherein the widthwise sealing unit includes: a top and bottom surface sealing unit that bonds a bottom surface of the stick container where the first filler is to be filled and bonds a top surface of the stick container where the second filler has been injected; and a separation surface sealing unit that bonds a central portion of the stick container after the first filer is filled and before the second filler is filled.

The top and bottom surface sealing unit is formed to be movable in the lengthwise direction.

The apparatus for manufacturing a stick container further comprises: a cutting unit that cuts the bottom surface or the top surface of the stick container, which has been bonded by the top and bottom surface sealing unit, along a widthwise direction.

The apparatus for manufacturing a stick container further comprises: a cooling/notch unit that is placed between the widthwise sealing unit and the cutting unit and configured to cool a portion bonded by the widthwise sealing unit or form a notch at the portion bonded by the widthwise sealing unit.

The cooling/notch unit is formed to be movable in the lengthwise direction.

The filling unit includes: a housing that extends in a lengthwise direction, a first filling unit that is inserted into the housing and sprays the first filler in liquid form; and a second filling unit that is inserted into the housing and injects the second filler in powder form.

According to another aspect of the present disclosure, A method for manufacturing a stick container having a pair of filler accommodating parts by processing a sheet-shaped material, the method comprises: a lengthwise sealing process for bonding side surfaces of the material to each other in order for the material to have an internal space which is elongated in a lengthwise direction; widthwise sealing processes for bonding the material to separate the internal space along the lengthwise direction; and filling processes for sequentially injecting the first filler and the second filler into the separated internal spaces, wherein the widthwise sealing processes includes: a top and bottom surface sealing process for bonding a bottom surface of the stick container where the first filler is to be filled and bonding a top surface of the stick container where the second filler has been filled; and a separation surface sealing process for bonding a central portion of the stick container after the first filer is filled and before the second filler is filled.

The method for manufacturing a stick container further comprises: a cutting process for cutting the bottom surface or the top surface of the stick container, which has been bonded in the top and bottom surface sealing process, along a widthwise direction.

The method for manufacturing a stick container further comprises: a cooling/notch process for cooling a portion bonded in the widthwise sealing processes or forming a notch at the bonded portion before the cutting process is performed.

The filling processes include: a process for spraying the first filler in liquid form to the stick container in which the bottom surface is bonded; and a process for injecting the second filler in powder form to the stick container in which the central portion is bonded.

A cosmetic container comprises the stick container, wherein the first filler is selected from the group consisting of sodium hyaluronate, retinol, retinyl palmitate, retinyl acetate, retinoic acid, coenzyme Q10, elastin, collagen, hyaluronic acid, ceramide, caffeine, chitosan, ascorbic acid, ascorbyl glucoside, alpha-Bisabolol, tocopherol, tocopheryl acetate, arbutin, niacinamide, adenosine, retinol acetate, natural extracts, synthetic oils, vegetable oils and combinations thereof, and the second filler includes one of vitamin A powder, vitamin B powder, vitamin C powder, vitamin D powder, vitamin E powder, vitamin F powder, ascorbic acid powder, glycolic acid powder, lactic acid powder, citric acid powder, salicylic acid powder, hydrolyzed collagen powder, erythritol powder, betaine powder, sodium hyaluronate powder (or hyaluronic acid sodium salt powder), trehalose powder, urea powder, dimethyl sulfone powder (or sulfur powder), allantoin powder, gluconic lactone powder, madecassoside powder, natural extract water-soluble pigment powder and natural extract oil-soluble pigment powder or powder selected from the group consisting of combinations thereof.

According to the present disclosure, contents different in component and effect are injected into a first filling part and a second filling part, respectively, of a single pack container and packed in order for a user to mix and use the contents right before use. Therefore, the effects of the contents can be maximized.

Also, according to the present disclosure, a stick container, which is filled with different kinds of contents in respective separated internal spaces, can be manufactured in great numbers at once.

### BRIEF DESCRIPTION OF THE DRAWINGS

In the detailed description that follows, embodiments are described as illustrations only since various changes and modifications will become apparent to a person with ordinary skill in the art from the following detailed description. The use of the same reference numbers in different figures indicates similar or identical items.

FIG. 1A and FIG. 1B illustrate a stick container manufactured by an apparatus and a method according to an embodiment of the present disclosure.

FIG. 2 illustrates an apparatus for manufacturing a stick container according to an embodiment of the present disclosure.

FIG. 3 illustrates a filling unit according to an embodiment of the present disclosure.

FIG. 4 illustrates a method for manufacturing a stick container according to an embodiment of the present disclosure.

### DETAILED DESCRIPTION

Hereinafter, embodiments of the present disclosure will be described in detail with reference to the accompanying drawings so that the present disclosure may be readily implemented by those skilled in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Further, the term “comprises or includes” and/or “comprising or including” used in the document means that one or more other components, steps, operation and/or existence or addition of elements are not excluded in addition to the described components, steps, operation and/or elements unless context dictates otherwise.

Hereinafter, embodiments of the present disclosure will be described in detail with reference to the accompanying drawings.

An apparatus for manufacturing a stick container according to an embodiment of the present disclosure is capable of manufacturing a stick container in great numbers at once by injecting contents different in component and effect into a first filling part and a second filling part, respectively, of a single pack container and packing them in order for a user to mix and use the contents right before use, and, thus the effects of the contents can be maximized.

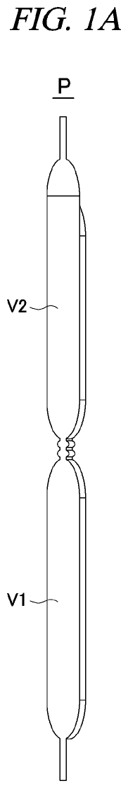

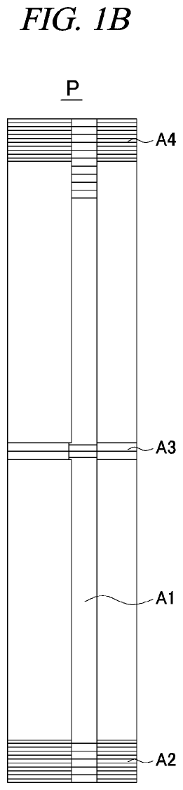

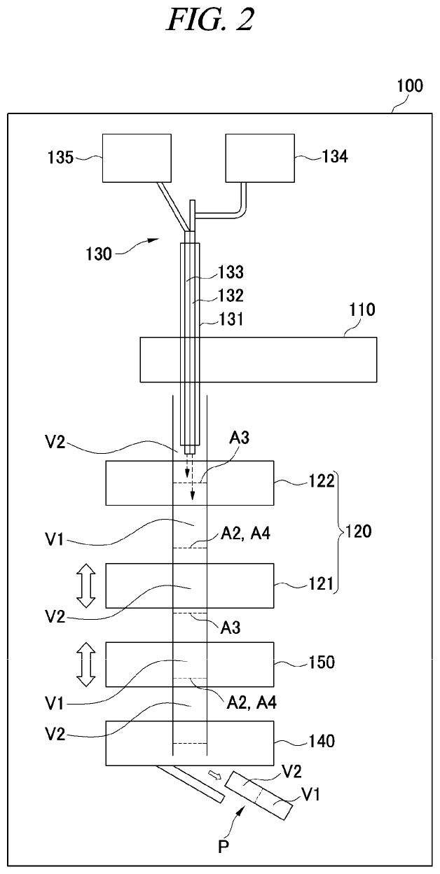

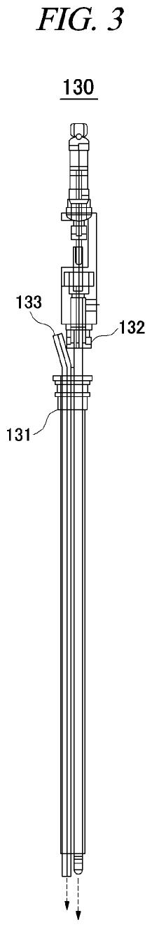

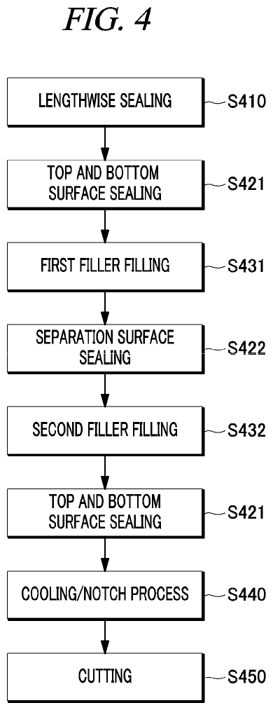

FIG. 1A and FIG. 1B illustrate a stick container manufactured by an apparatus and a method according to an embodiment of the present disclosure, and FIG. 2 illustrates an apparatus for manufacturing a stick container according to an embodiment of the present disclosure. FIG. 3 illustrates a filling unit according to an embodiment of the present disclosure, and FIG. 4 illustrates a method for manufacturing a stick container according to an embodiment of the present disclosure.

A stick container P of the present disclosure is a container packing contents therein and has a shape elongated in one direction (a lengthwise direction) and includes three bonding surfaces. Here, the three bonding surfaces may include a bonding surface (a side surface A1 to be described later) formed on the side or back of the container along the one direction and two bonding surfaces (a top surface A4 and a bottom surface A2 to be described later) sealing the top and the bottom, respectively, of the container.

More specifically, in the present disclosure, the stick container P refers to a container packing different kinds of contents which are individually separated, and includes a container manufactured by an apparatus and method for manufacturing a stick container according to the present disclosure. Here, the contents may include powdered and liquid ingredients, but may not be particularly limited thereto. For example, the powdered and liquid ingredients may include cosmetic ingredients effective in anti-wrinkle, skin whitening, UV blocking, anti-aging, moisturizing, skin tightening or the like and drugs for preventing and treating skin diseases and may provide effective effects on the skin.

The stick container P is processed from a sheet-shaped material and formed to have a pair of filler accommodating parts V1 and V2. For example, a liquid content may be accommodated in a first filler accommodating part V1 and a powdered content may be accommodated in a second filler accommodating part V2. As such, contents different in form from each other may be accommodated in the first filler accommodating part V1 and the second filler accommodating part V2, respectively.

The sheet-shaped material may be vinyl or film for packing. As shown in FIG. 1A, an internal space of the stick container P is divided vertically (lengthwise) into the first filler accommodating part V1 and the second filler accommodating part V2. For example, the first filler accommodating part V1 and the second filler accommodating part V2 may be filled with different kinds of contents, respectively.

Also, the stick container P may include the back surface or side surface A1, the bottom surface A2, a separation surface A3 and the top surface A4 as shown in FIG. 1B. The separation surface A3 of the stick container P divides the internal space of the stick container P into upper and lower spaces, which form a pair of filler accommodating parts V1 and V2.

Referring to FIG. 2, an apparatus 100 for manufacturing a stick container according to an embodiment of the present disclosure (hereinafter, referred to as “stick container manufacturing apparatus”) includes a lengthwise sealing unit 110, a widthwise sealing unit 120, a filling unit 130, a cutting unit 140 and a cooling/notch unit 150. The widthwise sealing unit 120 may further include a top and bottom surface sealing unit 121 and a separation surface sealing unit 122, and the filling unit 130 may further include a housing 131, a first filling unit 132 and a second filling unit 133.

The lengthwise sealing unit 110 may bond side surfaces of the material to each other in order for the material to have an internal space which is elongated in a lengthwise direction. Specifically, the lengthwise sealing unit 110 may bond both lengthwise side surfaces of the stick container P to each other to generate an internal space. The lengthwise side surfaces of the stick container P bonded to each other correspond to the back surface or side surface A1 of the stick container P as shown in FIG. 1B.

According to an embodiment of the present disclosure, the lengthwise sealing unit 110 may form the side surface A1 of the stick container P to generate an internal space along the lengthwise direction of the stick container P.

The widthwise sealing unit 120 may bond the material to separate the internal space along the lengthwise direction. The widthwise sealing unit 120 may bond widthwise side surfaces of the stick container P to seal the generated internal space. The widthwise side surfaces of the stick container P correspond to the bottom surface A2, the separation surface A3 and the top surface A4 as shown in FIG. 1B.

The widthwise sealing unit 120 may further include the top and bottom surface sealing unit 121 and the separation surface sealing unit 122 to form the bottom surface A2, the separation surface A3 and the top surface A4 of the stick container P. The top and bottom surface sealing unit 121 forms the bottom surface A2 and the top surface A4 of the stick container P, and the separation surface sealing unit 122 forms the separation surface A3.

According to an embodiment of the present disclosure, the widthwise sealing unit 120 bonds the bottom surface A2 of the stick container P where the first filler is to be filled and bonds the top surface A4 of the stick container P where the second filler has been injected.

After the first filer is filled and before the second filler is filled, the widthwise sealing unit 120 bonds a central portion of the stick container P. The central portion corresponds to the separation surface A3 that can separate the internal space of the stick container P. The separation surface A3 is formed to block the openness between the first filler accommodating part V1 and the second filler accommodating part V2, and, thus, the internal space of the stick container P is divided into the first filler accommodating part V1 and the second filler accommodating part V2.

According to an embodiment of the present disclosure, the separation surface A3 may be formed to break a bonded portion of the separation surface A3 when a user applies pressure to the stick container P right before using the contents accommodated in the filler accommodating parts V1 and V2 of the stick container P, and, thus, the first filler accommodated in the first filler accommodating part V1 and the second filler accommodated in the second filler accommodating part V2 can be mixed with each other. For example, a liquid ingredient accommodated in the first filler accommodating part V1 and a powered ingredient accommodated in the second filler accommodating part V2 may be mixed with each other.

More specifically, when the user presses the first filler accommodating part V1 to apply additional pressure to the first filler accommodating part V1 right before opening the stick container P, it is possible to clear the block between the first filler accommodating part V1 and the second filler accommodating part V2. For another example, if the user presses the second filler accommodating part V2 to apply additional pressure to the second filler accommodating part V2 in a state where the first filler accommodating part V1 of the stick container P is opened, the content in the first filler accommodating part V1 and the content in the second filler accommodating part V2 can be mixed with each other. Here, the contents in the filler accommodating parts V1 and V2 may be mixed at different mixing ratios by differentiating the intensity of pressure applied to the filler accommodating parts V1 and V2. More specifically, the amount of the powdered ingredient in the second filler accommodating part V2 to be mixed with the liquid ingredient in the first filler accommodating part V1 can be adjusted.

By mixing the first filler and the second filler separately filled in the respective filler accommodating parts V1 and V2 of the stick container P right before use or right before opening the stick container P, the effects of the contents can be maximized.

More specifically, in the stick container P including the side surface A1 formed by means of the lengthwise sealing unit 110, the bottom surface A2 is formed first by means of the top and bottom surface sealing unit 121 of the widthwise sealing unit 120 and the first filler is filled and then, the separation surface A3 is formed by means of the separation surface sealing unit 122 and the second filler is filled. Then, the top surface A4 is formed by means of the top and bottom surface sealing unit 121, and, thus, the internal space of the stick container P can be sealed. In the manufacturing process according to the present disclosure, the top surface A4 of any one stick container P can be the bottom surface A2 of another stick container P to be processed next.

According to an embodiment of the present disclosure, the top and bottom surface sealing unit 121 may be formed to be movable in the lengthwise direction.

For example, after forming the bottom surface A2 of the stick container P, the top and bottom surface sealing unit 121 may move vertically to form the top surface A4 of the stick container P and bond widthwise side surfaces of the stick container P and thus form the bottom surface A2 and the top surface A4.

The filling unit 130 may be configured to fill the first filler and the second filler in the separated internal spaces, respectively.

As described above, in the stick container P including the side surface A1, the bottom surface A2 may be formed by means of the top and bottom surface sealing unit 121 and the filling unit 130 may fill the first filler, and the separation surface A3 that seals the first filler and serves as the bottom surface of the second filler may be formed. Then, the top surface A4 capable of sealing the second filler is formed by means of the top and bottom surface sealing unit 121.

According to an embodiment of the present disclosure, the filling unit 130 may include the housing 131, the first filling unit 132 and the second filling unit 133. The filling unit 130 may further include a first storage unit 134 connected to the first filling unit 132 and a second storage unit 135 connected to the second filling unit 133. Referring to FIG. 3, the filling unit 130 includes the housing 131 that extends in a lengthwise direction, the first filling unit 132 that is inserted into the housing 131 and sprays the first filler in liquid form and the second filling unit 133 that is inserted into the housing 131 and injects the second filler in powder form.

Here, different kinds of contents may be contained in the first filling unit 132 and the second filling unit 133, respectively. The respective contents may be filled in the first filling unit 132 and the second filling unit 133, being separated from each other depending on ingredient and efficacy and then, the respective contents may be filled in the filler accommodating parts V1 and V2 of the stick container P.

The first filling unit 132 and the second filling unit 133 may be configured to inject different kinds of contents into the first filler accommodating part V1 and the second filler accommodating part V2, respectively, of the single stick container P. More specifically, the first filling unit 132 and the second filling unit 133 may be introduced into or withdrawn from the filler accommodating parts V1 and V2 of the stick container P. When the first filling unit 132 and the second filling unit 133 are completely introduced into the filler accommodating parts V1 and V2 and start to be withdrawn from the filler accommodating parts V1 and V2, the first filling unit 132 and the second filling unit 133 may inject predetermined amounts of the contents into the filler accommodating parts V1 and V2, respectively.

The first filling unit 132 and the second filling unit 133 may be configured as nozzles for injecting the contents into the filler accommodating parts V1 and V2, respectively. The first filling unit 132 and the second filling unit 133 may be connected to the first storage unit 134 and the second storage unit 135 that store the contents, respectively. For example, the first storage unit 134 connected to the first filling unit 132 may store the first filler in liquid form and the second storage unit 135 connected to the second filling unit 133 may store the second filler in powder form.

According to an embodiment of the present disclosure, the first storage unit 134 and the second storage unit 135 may be spaced apart from each other as shown in FIG. 2. The first storage unit 134 and the second storage unit 135 spaced apart from each other are connected to the first filling unit 132 and the second filling unit 133, respectively. The first filling unit 132 and the second filling unit 133 which are spaced apart from each other may be gathered from an upper end of the housing 131 and placed in parallel to each other inside the single housing 131.

As described above, the first filler accommodating part V1 and the second filler accommodating part V2 of the stick container P can be filled with different kinds of contents by means of the single housing 131. Thus, there is no need to provide a plurality of housings 131. For example, a liquid content can be filled in the first filler accommodating part V1 of the stick container P and a powdered content can be filled in the second filler accommodating part V2 by means of the single housing 131.

According to an embodiment of the present disclosure, in the stick container P including the bottom surface A2, the first filler is sprayed from the first filling unit 132, and in the stick container P including the separation surface A3, the second filler is sprayed from the second filling unit 133, and, thus, different kinds of contents can be individually separated and filled in the single stick container P.

The cutting unit 140 may cut the bottom surface A2 or the top surface A4 of the stick container P, which has been bonded by the top and bottom surface sealing unit 121, along a widthwise direction.

According to an embodiment of the present disclosure, the cutting unit 140 may be placed at the bottom of the stick container manufacturing apparatus 100 as shown in FIG. 2. The cutting unit 140 may cut the stick container P, which is completely manufactured by filling all the filler accommodating parts V1 and V2, along the widthwise direction to allow each separate stick container P to be discharged.

The bottom surface A2 or the top surface A4 of the stick container P filled with the first filler and the second filler in the filler accommodating parts V1 and V2, respectively, through the widthwise sealing unit 120 and the filling unit 130 may be cut by the cutting unit 140 along the widthwise direction to manufacture the single stick container P.

The cooling/notch unit 150 may be placed between the widthwise sealing unit 120 and the cutting unit 140 and configured to cool a portion bonded by the widthwise sealing unit 120 or form a notch at the portion bonded by the widthwise sealing unit 120. Here, the notch is a portion processed for convenience in use, such as opening the stick container P, and maybe a triangular mark or small defect.

For example, before each manufactured stick containers P is cut, the bonded portion may be cooled by the cooling/notch unit 150 to enhance the adhesive strength or to facilitate cutting of the top surface A4 or the bottom surface A2.

For another example, before each manufactured stick containers P is cut, a notch may be formed at the bonded portion by the cooling/notch unit 150 to facilitate easier opening of the stick container P.

According to an embodiment of the present disclosure, the cooling/notch unit 150 may be formed to be movable in the lengthwise direction. Therefore, the cooling/notch unit 150 can cool all the bonded portions of the side surface A1, the bottom surface A2, the separation surface A3 and the top surface A4 of the stick container P so as to enhance the adhesive strength.

According to an embodiment of the present disclosure, the stick container P includes a stick container P manufactured by means of the above-described stick container manufacturing apparatus 100.

Referring to FIG. 4, a method for manufacturing a stick container according to an embodiment of the present disclosure includes a lengthwise sealing process S410, widthwise sealing processes S421 and S422, filling processes S431 and S432, a cutting process S450, and a cooling/notch process S440.

The widthwise sealing processes S421 and S422 may include a top and bottom surface sealing process S421 and a separation surface sealing process S422, and the filling processes S431 and S432 may include a first filler spraying process S431 and a second filler injecting process S432.

In the lengthwise sealing process S410, side surfaces of a sheet-shaped material may be bonded to each other in order for the material to have an internal space which is elongated in a lengthwise direction. More specifically, in the lengthwise sealing process S410, both lengthwise side surfaces of the stick container P are bonded to each other by means of the lengthwise sealing unit 110 to form the side surface A1.

In the widthwise sealing processes S421 and S422, the material may be bonded to separate the internal space along the lengthwise direction. In the widthwise sealing processes S421 and S422, widthwise side surfaces of the stick container P are bonded by means of the widthwise sealing unit 120 to form the bottom surface A2, the separation surface A3 and the top surface A4.

According to an embodiment of the present disclosure, the widthwise sealing processes S421 and S422 include the top and bottom surface sealing process S421 for bonding the bottom surface A2 of the stick container P where the first filler is to be filled and bonding the top surface A4 of the stick container P where the second filler has been filled and the separation surface sealing process S422 for bonding a central portion of the stick container P after the first filer is filled and before the second filler is filled. The central portion corresponds to the separation surface A3 that can separate the internal space of the stick container P. If the separation surface A3 is formed, the internal space of the stick container P can be divided into the first filler accommodating part V1 and the second filler accommodating part V2. For example, a liquid content may be accommodated in the first filler accommodating part V1 and a powdered content may be accommodated in the second filler accommodating part V2. As such, contents different in form from each other may be accommodated in the first filler accommodating part V1 and the second filler accommodating part V2, respectively.

As described above, the separation surface A3 may be formed to break a bonded portion of the separation surface A3 when a user applies pressure to the stick container P right before using the contents accommodated in the filler accommodating parts V1 and V2 of the stick container P, and, thus, the first filler accommodated in the first filler accommodating part V1 and the second filler accommodated in the second filler accommodating part V2 can be mixed with each other. For example, a liquid ingredient accommodated in the first filler accommodating part V1 and a powered ingredient accommodated in the second filler accommodating part V2 may be mixed with each other, and the amount of the powdered ingredient in the second filler accommodating part V2 to be mixed with the liquid ingredient in the first filler accommodating part V1 can be adjusted.

By mixing the first filler and the second filler separately filled in the respective filler accommodating parts V1 and V2 of the stick container P right before use, the effects of the contents can be maximized.

In the filling processes S431 and S432, the first filler and the second filler may be sequentially injected into the separated internal spaces of the stick container P.

According to an embodiment of the present disclosure, the filling processes S431 and S432 may include a process S431 for spraying the first filler in liquid form to the stick container P in which the bottom surface A2 is bonded and a process S432 for injecting the second filler in powder form to the stick container P in which the central portion is bonded. According to an embodiment of the present disclosure, different kinds of contents can be filled in the filler accommodating parts V1 and V2, respectively, of the single stick container P. First, the first filler is injected by means of the first filling unit 132 of the stick container P into the stick container P where the bottom surface A2 is bonded, and the separation surface A3 that seals the first filler and serves as the bottom surface of the second filler is formed. Then, the second filler is injected by means of the second filling unit 133. The different kinds of contents can be individually separated and filled in the filler accommodating parts V1 and V2 of the single stick container P. The first filling unit 132 may be connected to the first storage unit 134, and the second filling unit 133 may be connected to the second storage unit 135. Different kinds of contents may be accommodated in the first storage unit 134 and the second storage unit 135, respectively. For example, a liquid content may be stored in the first storage unit 134 and a powdered content may be stored in the second storage unit 135.

In the cooling/notch process S440, a portion bonded in the widthwise sealing processes S421 and S422 may be cooled or a notch may be formed at the bonded portion before the cutting process S450 is performed.

More specifically, before the stick containers P subjected to the sealing processes S410, S421 and S422 and the filling processes S431 and S432 is cut, the bonded portion may be cooled to enhance the adhesive strength or to facilitate cutting of the stick containers P.

Further, before the stick containers P subjected to the sealing processes S410, S421 and S422 and the filling processes S431 and S432 is cut, a notch may be formed at the bonded portion to facilitate easier opening of the stick container P.

In the cutting process S450, the bottom surface A2 or the top surface A4 of the stick container P, which has been bonded in the top and bottom surface sealing process S421, may be cut along a widthwise direction.

According to an embodiment of the present disclosure, the bottom surface A2 and the top surface A4 of the stick container P, which is completely manufactured through the sealing units 110 and 120 and the filing unit 130, are cut by the cutting unit 140 to allow each separate stick container P to be discharged.

According to an embodiment of the present disclosure, the stick container P includes a stick container P manufactured by the above-described stick container manufacturing method. In the stick container P, a pair of filler accommodating parts V1 and V2 can be filled with different contents, respectively.

The first filler or the second filler which can be filled in the first filler accommodating part V1 or the second filler accommodating part V2, respectively, may be selected from the group consisting of sodium hyaluronate, retinol, retinyl palmitate, retinyl acetate, retinoic acid, coenzyme Q10, elastin, collagen, hyaluronic acid, ceramide, caffeine, chitosan, ascorbic acid, ascorbyl glucoside, alpha-Bisabolol, tocopherol, tocopheryl acetate, arbutin, niacinamide, adenosine, retinol acetate, natural extracts, synthetic oils, vegetable oils and combinations thereof, but may not be limited thereto.

Also, the second filler which can be filled in the second filler accommodating part V2 may include one of vitamin A powder, vitamin B powder, vitamin C powder, vitamin D powder, vitamin E powder, vitamin F powder, ascorbic acid powder, glycolic acid powder, lactic acid powder, citric acid powder, salicylic acid powder, hydrolyzed collagen powder, erythritol powder, betaine powder, sodium hyaluronate powder (or hyaluronic acid sodium salt powder), trehalose powder, urea powder, dimethyl sulfone powder (or sulfur powder), allantoin powder, gluconic lactone powder, madecassoside powder, natural extract water-soluble pigment powder and natural extract oil-soluble pigment powder or powder selected from the group consisting of combinations thereof, but may not be limited thereto.

The natural extracts may include, for example, aloe, green tea, ginseng, red ginseng, pearl, pyroligneous liquor, pine needle, ginko leaf, propolis, snail mucus, caviar, broccoli, blueberry, chlorella, mangosteen, guava, Cornus officinalis, salmon roe, raspberry, wild berry, edelweiss, chamomile, lavender, peppermint, eucalyptus, lemon balm, oregano, tea tree, Houttuynia cordata, citron and the like, but may not be limited thereto. The synthetic oils may include mineral oil, caprylic/capric triglyceride, dimethicone, cyclomethicone, cetyl ethylhexanoate and the like, but may not be limited thereto. The vegetable oils may include moringa oil, avocado oil, Jojoba seed oil, apricot seed oil, macadamia seed oil, walnut oil, green tea seed oil, sunflower seed oil, camellia seed oil and the like, but may not be limited thereto. The liquid ingredient may further include a functional additive, and the functional additive may be, for example, fragrance, preservative, moisturizer and the like, but may not be limited thereto.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

1. An apparatus for manufacturing a stick container having a pair of filler accommodating parts by processing a sheet-shaped material, the apparatus comprising:
a lengthwise sealing unit that bonds side surfaces of the material to each other in order for the material to have an internal space which is elongated in a lengthwise direction;
a widthwise sealing unit that bonds the material to separate the internal space along the lengthwise direction; and
a filling unit that is configured to fill a first filler and a second filler in the separated internal spaces, respectively,
wherein the widthwise sealing unit includes:
a top and bottom surface sealing unit that bonds a bottom surface of the stick container where the first filler is to be filled and bonds a top surface of the stick container where the second filler has been injected; and
a separation surface sealing unit that bonds a central portion of the stick container after the first filer is filled and before the second filler is filled.

2. The apparatus for manufacturing a stick container of claim 1,
wherein the top and bottom surface sealing unit is formed to be movable in the lengthwise direction.

3. The apparatus for manufacturing a stick container of claim 1, further comprising:
a cutting unit that cuts the bottom surface or the top surface of the stick container, which has been bonded by the top and bottom surface sealing unit, along a widthwise direction.

4. The apparatus for manufacturing a stick container of claim 3, further comprising:
a cooling/notch unit that is placed between the widthwise sealing unit and the cutting unit and configured to cool a portion bonded by the widthwise sealing unit or form a notch at the portion bonded by the widthwise sealing unit.

5. The apparatus for manufacturing a stick container of claim 4,
wherein the cooling/notch unit is formed to be movable in the lengthwise direction.

6. The apparatus for manufacturing a stick container of claim 1,
wherein the filling unit includes:
a housing that extends in a lengthwise direction,
a first filling unit that is inserted into the housing and sprays the first filler in liquid form; and
a second filling unit that is inserted into the housing and injects the second filler in powder form.

7. A method for manufacturing a stick container having a pair of filler accommodating parts by processing a sheet-shaped material, the method comprising:
a lengthwise sealing process for bonding side surfaces of the material to each other in order for the material to have an internal space which is elongated in a lengthwise direction;
widthwise sealing processes for bonding the material to separate the internal space along the lengthwise direction; and
filling processes for sequentially injecting the first filler and the second filler into the separated internal spaces,
wherein the widthwise sealing processes includes:
a top and bottom surface sealing process for bonding a bottom surface of the stick container where the first filler is to be filled and bonding a top surface of the stick container where the second filler has been filled; and
a separation surface sealing process for bonding a central portion of the stick container after the first filer is filled and before the second filler is filled.

8. The method for manufacturing a stick container of claim 7, further comprising:
a cutting process for cutting the bottom surface or the top surface of the stick container, which has been bonded in the top and bottom surface sealing process, along a widthwise direction.

9. The method for manufacturing a stick container of claim 8, further comprising:
a cooling/notch process for cooling a portion bonded in the widthwise sealing processes or forming a notch at the bonded portion before the cutting process is performed.

10. The method for manufacturing a stick container of claim 7,
wherein the filling processes include:
a process for spraying the first filler in liquid form to the stick container in which the bottom surface is bonded; and
a process for injecting the second filler in powder form to the stick container in which the central portion is bonded.

11. A stick container manufactured by means of an apparatus for manufacturing a stick container of claim 1.

12. A stick container manufactured by a method for manufacturing a stick container of claim 7.

13. A cosmetic container, comprising:
container of claim 11,
wherein the first filler is selected from the group consisting of sodium hyaluronate, retinol, retinyl palmitate, retinyl acetate, retinoic acid, coenzyme Q10, elastin, collagen, hyaluronic acid, ceramide, caffeine, chitosan, ascorbic acid, ascorbyl glucoside, alpha-Bisabolol, tocopherol, tocopheryl acetate, arbutin, niacinamide, adenosine, retinol acetate, natural extracts, synthetic oils, vegetable oils and combinations thereof, and
the second filler includes one of vitamin A powder, vitamin B powder, vitamin C powder, vitamin D powder, vitamin E powder, vitamin F powder, ascorbic acid powder, glycolic acid powder, lactic acid powder, citric acid powder, salicylic acid powder, hydrolyzed collagen powder, erythritol powder, betaine powder, sodium hyaluronate powder (or hyaluronic acid sodium salt powder), trehalose powder, urea powder, dimethyl sulfone powder (or sulfur powder), allantoin powder, gluconic lactone powder, madecassoside powder, natural extract water-soluble pigment powder and natural extract oil-soluble pigment powder or powder selected from the group consisting of combinations thereof.
